(19) United States
(12) Patent Application Publication
(10) Pub. No.: US 2007/0270801 A1
(43) Pub. Date: Nov. 22, 2007

(54) Adapter, a fixation pin and a method for fixation of a supporting structure to a body part

(75) Inventors: Thomas Arn, Lidingo (SE); Bo Nilsson, Osterskar (SE)

Correspondence Address: BUCHANAN, INGERSOLL & ROONEY PC, POST OFFICE BOX 1404, ALEXANDRIA, VA, 22313-1404, US

(73) Assignee: ELEKTA AB (publ), Stockholm (SE)

(21) Appl. No.: 11/412,837

(22) Filed: Apr. 28, 2006

(51) Int. Cl. A61B 17/00

(52) U.S. Cl. 606/54

(57) ABSTRACT

The invention relates to an adapter (5) for a fixation pin (2) of the type which is adapted for fixating a supporting structure to a bony structure of a human or animal body part, and which is provided with a shank portion (4). The adapter (5) comprises a sharp-pointed tip (7), for penetration into the bony structure of the body part, and an engagement formation (8) for removable connection to an inner end of said shank portion (4). The invention also relates to a fixation pin and a method for fixating a supporting structure to a human or animal body part.

The invention relates to an adapter for a fixation pin of the type which is adapted for fixating a supporting structure to a bony structure of a human or animal body part, said fixation pin comprising a shank portion and being displaceably mounted in the supporting structure such that an inner end of the shank portion can be advanced towards or retracted from the body part to which the supporting structure is to be fixated.

The invention also relates to a fixation pin as well as a method for fixation of such a supporting structure to a bone of a human or animal body part.

### BACKGROUND OF THE INVENTION

During treatment or diagnosis of humans or animals, it is in many cases common practice to attach some kind of supporting structure to the bone or bony structure of the body part to be treated or examined. This can be done for purpose of fixation of two body parts in relation to each other, for fixation of a body part in relation to a treating or diagnosing apparatus or for attaching a treating or diagnosing instrument to a body part. The supporting structure is fixated to the body part by means of fixation pins, which each are formed with a sharp-pointed tip and usually are threaded to be screwed into a threaded hole in the supporting structure. By screw rotating the fixation pins, the sharp-pointed tips of the fixation pins can be displaced towards the body part to be fixated, such that the tip of each fixation pin penetrates through a layer of tissue, if present, and a small distance into the bone. At least three fixation pins are required, which penetrates into the body part from different directions, to fixate the body part and the supporting structure rigidly in relation to each other in all directions.

One area for diagnosis or treatment of a human body, where this technique is frequently utilized, is when diagnosing or treating the human brain. In this case use is made of a so called stereotactic frame, which is fixated to the skull by means of fixation pins and it is extremely important that the skull and the frame is rigidly fixated in relation to each other, such that the proper target area of the brain is diagnosed or treated to avoid incorrect diagnosis or treatment. The diagnosis in this case may comprise Magnetic (Resonance-Imaging (MR-imaging), Computerized Tomography Imaging (CT-imaging) or selection of samples by means of biopsy needles. The treatment, on the other hand, may comprise e.g. radiation surgery. When performing tomography or radiation surgery, the frame is used to fixate the head rigidly, in relation to the apparatus which is used, and in a well defined and corresponding position in the imaging apparatus as well as in the radiation surgery apparatus. When performing selection of samples, on the other hand, the frame is used to fixate the instruments to be used in relation to the head to ensure selection of samples of the correct, target area of the brain.

Today such supporting structures are fixated to the patient with fixation pins made of preferable aluminium but also titanium. Other materials like different kinds of plastics are commercially available, but they tend to be less rigid than the ones made of metal. Tips are either made of the base pin material (aluminium or titanium) or a harder material, such as hard metal or ceramic, to ensure the tip sharpness after multiple usage. Most of the commercially available pins are reusable but there are single use pins made of sole aluminium. However, there are different problems with the fixation pins used today.

When MR-imaging, pins made of conducting material tend to induce RF-energy (Radio Frequency) at the tip which has caused burn injuries of patients. Titanium seem to induce more RF-energy than aluminium. This problem is not present when using plastic pins as long as the pins does not contain conducting fillers or fibres like carbon fibres. Another common problem in MR-imaging is artefact or distortion problems of images or attenuation of the signals, which is caused by magnetic trace material in mainly hard metal tips and titanium.

In CT-imaging, the pins causes artefacts that originates from geometry and density of the in material. Hard metal and ceramic are high density materials where this problem is significant.

In the past years, the cleaning and sterilization recommendations at the hospitals have tend to go towards longer cycles, higher temperatures and more aggressive chemicals to reduce the risk of serious diseases. Plastic reusable pins are usually more sensitive to these methods and the material will degrade in an uncontrolled way that may be hazardous to the patient. There are not many commercial plastic pins left on the market today.

Sterility and cleanness is also a concern for pins made of two or more materials, e.g. body of aluminium and tip of hard metal. Autoclaving in high temperatures have shown to induce cracking of the pin bodies due to different characteristics of the materials.

The cost for manufacturing pins having an integrated tip of a different material, are much more expensive than for homogenous pins with tip and shank in the same material. However, pins of only one material can not be manufactured in a material that have a tip strength that makes the pins fully reusable and the cost for using these pins as disposables will be too expensive.

U.S. Pat. No. 5,643,268 discloses a fixation pin in form of a screw for fixation of a stereotactic frame on a head. The pin shaft is formed of reinforced plastics having a tip of semiprecious stone material. The pin is not adapted to be disposable but should be rigid enough to be used over and over again. However, plastics have a lower rigidity in comparison to aluminium and titanium. Accordingly, the pin diameter must be increased which has to result that the pins will not fit into existing stereotactic frames made for metallic pins. Moreover, this pin has not the same hygienic advantages as single use pins. There is also mechanical problems that can arise when combining two materials having different characteristics such that it will not hold for autoclaving as well as other, chemical sterilizing methods.-This together with the choose of tip will probably result in high costs for this solution.

### SUMMARY OF THE INVENTION

The object of the invention is to provide an adapter, which will reduce the total costs for manufacturing and using fixation pins for fixation of a supporting structure to a body part. At least this object will be achieved by an adapter for a fixation pin of the type which is adapted for fixating a supporting structure to a bony structure of a human or animal body part, and which is provided with a shank portion, the adapter comprising a sharp-pointed tip for penetration into the bony structure of the body and an engagement formation for removable connection to an inner end of said shank portion.

The invention also relates to a fixation pin and a method for fixation of a support structure to a body part, having essentially the same object as above. At least this object is achieved by a fixation pin for fixation of a supporting structure to a bony structure of a human or animal body part, comprising a shank portion and a detachable adapter having a sharp-pointed tip, for penetration into the bony structure, and a complementary engagement formation for connection to an inner end of the shank portion. The object is also achieved by a method for fixation of a supporting structure to a bony structure of a body part on a human or animal body, comprising the steps of:

providing a shank portion and mount it displaceably in the supporting structure;

providing a sterilized adapter having a sharp-pointed tip and attach it to an inner end of the shank portion; and

advancing the shank portion inwards such that the sharp-pointed tip of the adapter will penetrate into the bone for fixation of the supporting structure td the body part.

The invention is thus based on the understanding that the above-mentioned object can be achieved by providing the fixation pin with an adapter in its inner end, which comprises a sharp-pointed tip on one side, to allow penetration into the bony structure to a certain extent, and an engagement formation on the other side to allow connection to the inner end of the shank portion of the fixation pin.

In this way several advantages may be achieved. The fixation pin can be manufactured of a homogenous and durable material, e.g. a metal, which will enable the fixation pin to be manufactured to a low cost compared to a fixation pin having integrated tip and shank portions of different materials. The adapter can be made of a low cost material, having a strength and stiffness high enough for performing at least one fixation and subsequently the adapter can be disposed off. The shank portion, on the other hand, can be made of a more durable, and hence more expensive material and reused over and over again. The adapter, which will have contact with tissue and therefore possesses the largest risk to transfer infection to the patient, can be delivered thoroughly sterilized, contained in a sterile package, whereas the shank portion can be sterilized by autoclaving and/or chemicals and consist of a material that withstands repeated sterilization. In this way it is, in case there is desirable to have different materials in the adapter and the shank portion, possible to make the shank portion of a homogeneous material which will eliminate the risk of forming of cracks in the fixation pin during sterilization. It is possible to form the adapter of a polymeric or plastic material or other electrical and heat insulating material, which during MR-imaging will reduce artefact or distortion of the images and attenuation, and protect the patient against excessive heat generated by RF-energy during imaging, while the shank portion can be formed of a more strong and durable material. It is possible to form the adapter of a low density material, such as a polymeric or plastic material, which during CT-imaging will decrease artefacts and distortion of the images. The adapter can be manufactured by a low cost method, e.g. by injection moulding of a plastic material, which will ensure a low total cost for each adapter. An adapter, which will give the above advantages, can easily be adapted to fit on existing fixation pins without the need for replacing existing equipment.

Within the general idea, the invention can be realized in many different ways. Normally, it is preferred to form the adapter of a polymeric or plastic material, such as PEEK, PAI, PEI or other polymers commonly used in medical devices, which is comparatively cheap, can easily be formed to a low cost, is heat insulating and electrically insulating, at least if it doesn't contain any electrically conducting fillers or fibres like carbon fibres. The shank portion, on the other hand, is preferably formed of a strong and durable metallic material, such as aluminium or titanium, which can be sterilized and reused over and over again.

However, it is also conceivable to form the adapter of e.g. ceramics or a low cost metallic material, such as aluminium, which easily can be formed in the desired shape. At least in e.g. CT-imaging, radiation surgery or taking of samples, adapters of aluminium would not cause any disadvantages since it has low density and no RF-energy is generated during the imaging. In such case, the adapter could be formed as a disposable in for example low cost aluminium, whereas the fixation pin is formed of more strong and durable aluminium or titanium. When used in MR-imaging, an adapter of aluminium could be a bit more problematic since it conducts electricity and heat very well. However, this problem could be reduced by means of e.g. a plastic bushing, which insulates the fixation pin from the supporting structure, as is disclosed in WO03094769.

Moreover, instead of forming the shank portion of a metal, it could also be formed of a polymeric or plastic material. In such a case the adapter could be formed of for example disposable, low cost plastic, whereas the shank portion is formed of a more high performance plastic having e.g. reinforcing carbon fibres. However, plastics are in general weaker and less durable metals, which normally requires larger dimensions for a plastic shank portion to ensure proper function during repeated use.

The shank portion of the fixation pin is normally provided with threads, such that the displacement of the inner end of the fixation pin towards and away from the body part, is performed by screw rotating the shank portion through a threaded hole in the supporting structure. However, the displacement of the fixation pin could also be accomplished by other means than threads. Moreover, the adapter can be unrotatably attached to the shank portion. However, an additional advantage is achieved if the adapter is rotatably attached to the shank portion. In this case the damages on the tissue around the fixating point can be reduced if the tip portion of the adapter penetrates through the tissue without rotating. Instead, the adapter is rotating in relation to the shank portion.

The inventive adapter and fixation pin will hereinafter be described when used for fixating a stereotactic frame to the head of a patient. However, it should be understood that the invention could be used to fixate also other types of supporting structures to also other human or animal body parts.

### BRIEF DESCRIPTION OF THE DRAWINGS

In the single drawing is shown in:

FIG. 1 a perspective view of a stereotactic frame attached to a human head by means of fixation pins and adapters according to the invention,

FIG. 2 an exploded perspective view of fixation pin comprising a shank portion and an adapter; and

FIG. 3-7 longitudinal sections of adapters and the inner ends of the shank portions according to different embodiments.

### DETAILED DESCRIPTION OF EMBODIMENTS OF THE INVENTION

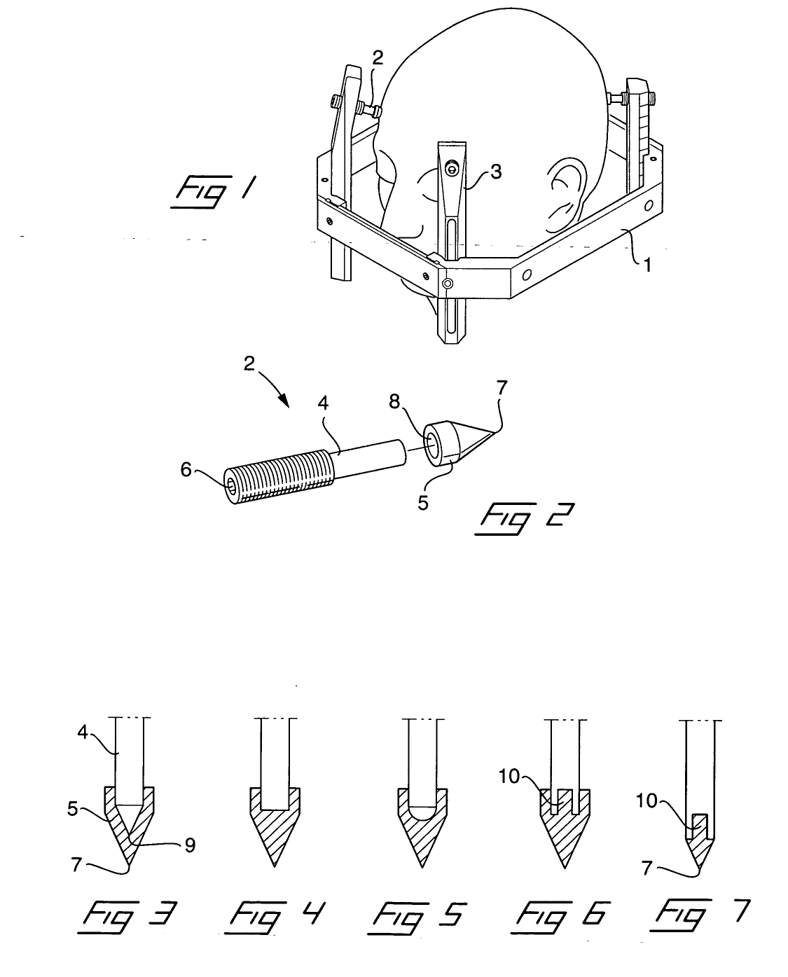

In FIG. 1 is shown an example of a supporting structure attached to a body part. In this case the supporting structure is a so called stereotactic frame 1, which is attached to a head of a patient by means of four fixation pins 2, of which only three is visible in the drawing. The stereotactic frame 1 is ring shaped and has four upward directed posts 3, which are adjustable in relation to the frame 1. In the upper end of each post 3, a fixation pin 2 is screwed into a threaded hole in the post such that a sharp-pointed tip of each fixation pin penetrates a small distance into the tissue and the skull and rigidly attaches the frame to the head.

FIG. 2 shows an exploded perspective view of an exemplary embodiment of a fixation pin 2 according to the invention. The fixation pin comprises a shank portion 4 and an adapter 5, of which the latter is detachable mountable to an inner end of the shank portion. As can be seen, the shank portion 4, in this embodiment, is formed with a screw thread in an outer portion and a smooth portion, having no screw thread, in an inner portion. In the outer end, the shank portion is formed with an engagement formation 6 for allowing screw rotation of the shank portion by means of a tool. The adapter 5 is formed with a sharp-pointed tip 7 in one end and an engagement formation 8, in form of a blind hole, in the other end, to allow detachable mounting to the shank portion 4 by simply slipping the hole 8 of the adapter over the inner end of the shank portion.

In the FIGS. 3-7 is shown examples of conceivable embodiments to accomplish the detachable connection between the adapter 5 and the shank portion 4.

FIG. 3 illustrates attaching of an adapter to an existing fixation pin, which previously has been used without any adapter and which for this reason is formed with a sharp-pointed tip 9.

FIG. 4-7, on the other hand, relates to embodiments where the shank portion 4 is formed without any sharp-pointed tip and accordingly is especially designed to be used together with an adapter 5. FIG. 4 corresponds to the embodiment in FIG. 2 and here the shank portion 4 is formed with an abrupt cut inner end and a correspondingly shaped hole in the adapter. In FIG. 5 the shank portion is formed with a rounded end. In FIG. 6 the inner end of the shank portion is provided with an recess into which a projection 10 in the adapter extends. The embodiment in FIG. 7 is similar to the embodiment of FIG. 6, except that the dimensions of the adapter is reduced such that the oblique surfaces of the adapter, which defines the tip 7, is terminated flush with the outer surfaces of the shank portion 4.

Normally, it is sufficient if the adapter 5 is mounted on the shank portion 4 by press fit since the adapter is pressed towards the shank portion upon abutment against and penetration into the body part. However, it would of course also be possible to arrange some kind of more secure engagement between the adapter and the shank portion. If for example the threads of the shank portion extends as far as to the inner end, the hole in the adapter can be provided with threads and screwed onto the shank portion. Likewise, the projection 10 in the adapter, could be provided with external treads which engages internal threads in the recess in the shank portion. However, as mentioned before, it can be advantageous to allow rotation of the adapter in relation to the shank portion to reduce damages to the tissue.

1. An adapter for a fixation pin of the type which is adapted for fixating a supporting structure to a bony structure of a human or animal body part, and which is provided with a shank portion, the adapter comprising a sharp-pointed tip for penetration into the bony structure of the body and an engagement formation for removable connection to an inner end of said shank portion.

2. An adapter according to claim 1, wherein the adapter is formed of a polymeric or plastic material.

3. An adapter according to claim 1, wherein the adapter is formed of a material different from the material in the shank portion.

4. An adapter according to claim 1, wherein the adapter is designed to be rotatable in relation to the shank portion.

5. An adapter according to claim 1, wherein the adapter is designed to be attached to a shank portion having a sharp-pointed tip.

6. An adapter according to claim 1, wherein the adapter is designed to be attached to a shank portion having no sharp-pointed tip.

7. A fixation pin for fixation of a supporting structure to a bony structure of a human or animal body part, comprising a shank portion and a detachable adapter having a sharp-pointed tip, for penetration into the bony structure, and a complementary engagement formation for connection to an inner end of the shank portion.

8. A fixation pin according to claim 7, wherein the adapter is formed of a polymeric or plastic material.

9. A fixation pin according to claim 7, wherein the adapter is formed of a material different from the material in the shank portion.

10. A fixation pin according to claim 7, wherein the adapter is rotatable in relation to the shank portion.

11. A fixation pin according to claim 7, wherein the adapter is designed to be attached to a shank portion having a sharp-pointed tip.

12. A fixation pin according to claim 7, wherein the adapter is designed to be attached to a shank portion having no sharp-pointed tip.

13. A fixation pin according to claim 7, wherein the shank portion is threaded.

14. A method for fixation of a supporting structure to a bony structure of a body part on a human or animal body, comprising the steps of:
providing a shank portion and mount it displaceably in the supporting structure;
providing a sterilized adapter having a sharp-pointed tip and attach it to an inner end of the shank portion; and
advancing the shank portion inwards such that the sharp-pointed tip of the adapter will penetrate into the bone for fixation of the supporting structure to the body part.

15. Method according to claim 14, comprising the further step of providing an adapter formed of a polymeric or plastic material.

16. Method according to claim 14, comprising the further step of mounting the adapter rotatably on the shank portion.

17. Method according to claim 14, comprising the further step of providing a shank portion having threads.

18. Method according to claim 14, comprising the further step of attaching an adapter to a shank portion having a sharp-pointed tip.

19. Method according to claim 14, comprising the further step of attaching the adapter to a shank portion having no sharp-pointed tip.
